(19) United States
(12) Patent Application Publication
(10) Pub. No.: US 2015/0322500 A1
(43) Pub. Date: Nov. 12, 2015

(54) Multiplexed diagnostic platform for point-of care pathogen detection

(71) Applicant: Lawrence Livermore National Security, LLC, Livermore, CA (US)

(72) Inventors: Mary Teresa McBride, Brentwood, CA (US); Benjamin J. Hindson, Livermore, CA (US); Steve B. Brown, Livermore, CA (US)

(21) Appl. No.: 14/454,433

(22) Filed: Aug. 7, 2014

Related U.S. Application Data
(63) Continuation of application No. 11/335,074, filed on Jan. 18, 2006, now abandoned.
Application No. 11/335,074 is a continuation-in-part of application No. 12/786,248, filed on May 24, 2010, now Pat. No. 9,052,255.
Application No. 12/786,248 is a division of application No. 10/643,797, filed on Aug. 19, 2003, now abandoned.
(60) Provisional application No. 60/406,159, filed on Aug. 26, 2002.

(51) Int. Cl. C12Q 1/68; B01L 7/00

(52) U.S. Cl. CPC C12Q 1/6837; B01L 7/52

(57) ABSTRACT

The invention provides a system for high-throughput multiplex analysis of target samples. A sample and reagent delivery unit is operatively connected to a thermal cycler for amplification of target nucleic acids. Microspheres are hybridized to the resulting amplicons in the thermal cycler. A flow cytometer is operatively connected to the thermal cycler or optionally a bead trap for washing the microspheres.

### CROSS REFERENCE TO RELATED APPLICATIONS

This application is a continuation of co-pending U.S. application Ser. No. 11/335,074, filed Jan. 18, 2006, which is herein incorporated in its entirety by reference.

This application is also a continuation-in-part of co-pending U.S. application Ser. No. 12/786,248, filed May 24, 2010, which is a divisional of abandoned U.S. application Ser. No. 10/643,797, filed Aug. 19, 2003, which claims the benefit of U.S. Provisional Application No. 60/406,159, filed Aug. 26, 2002, which is hereby incorporated in its entirety by reference.

### STATEMENT REGARDING FEDERALLY SPONSORED RESEARCH OR DEVELOPMENT

This invention was made with government support under Contract No. W-7405-ENG-48 between the U.S. Department of Energy and the University of California, for the operation of Lawrence Livermore National Laboratory. The government has certain rights in the invention.

### BACKGROUND OF THE INVENTION

Assays and devices for high-throughput analysis of targets have gained popularity of late. Given the threat of biological warfare agents, there is a significant need for the development of systems capable of automated and rapid analysis of samples for the detection of target organisms or agents.

While there has been marked improvement in such devices, the field is still reliant on large devices that are not conveniently moved from one location to another. In fact, many conventional high throughput detectors are simply too big to be portable or they are only capable of performing one of a series of required steps. There are clearly benefits of smaller devices. Not only are they available for detection of bioterror threats, but they would find use as point of care analyzers in hospitals and/or physicians' offices. However, to date, the systems are simply not amenable to reduction in size sufficient to make them truly portable.

Thus, there exists a critical need to develop convenient, accurate and easy-to-use point of care diagnostic and epidemiologic assessment tools for use in public health laboratories, hospitals and in the field. In addition, there is a significant need to develop portable or point-of-care multiplex detection capabilities. Currently, there are no available diagnostic instruments in use in hospital emergency rooms or clinics that can conduct rapid, sensitive, specific tests for common pathogens at “point-of-care”.

In addition, there is a need for apparatuses and/or distributed biothreat agent sensor networks that can operate in civilian applications. To operate in “Detect to Protect/Warn” type detection architectures, these platforms need to have several key properties. They need to be capable of detecting pathogens within a 1-2 hour time window, allowing for enough time to respond to an event. They need to be extremely low cost to maintain, since continuous monitoring is essential for many applications. These platforms need to have sufficient sensitivity to cover a broad geographical area (limiting the necessary number of sensors) and have sufficient selectivity to virtually eliminate false positives and false negatives. Currently available bio-weapons detection systems are designed primarily for military use on the battlefield. These systems are often expensive to deploy and ultimately unsuited for civilian protection.

In an article titled, “U.S. Is Deploying a Monitor System for Germ Attacks,” by Judith Miller in The New York Times on Jan. 22, 2003, it was reported, “To help protect against the threat of bioterrorism, the Bush administration on Wednesday will start deploying a national system of environmental monitors that is intended to tell within 24 hours whether anthrax, smallpox and other deadly germs have been released into the air, senior administration officials said today. The system uses advanced data analysis that officials said had been quietly adapted since the September 11 attacks and tested over the past nine months. It will adapt many of the Environmental Protection Agency's 3,000 air quality monitoring stations throughout the country to register unusual quantities of a wide range of pathogens that cause diseases that incapacitate and kill . . . . The new environmental surveillance system uses monitoring technology and methods developed in part by the Department of Energy's national laboratories. Samples of DNA are analyzed using polymerase chain reaction techniques, which examine the genetic signatures of the organisms in a sample, and make rapid and accurate evaluations of that organism . . . . ”

In an article titled, “Biodetectors Evolving, Monitoring U.S. Cities,” by Sally Cole in the May 2003 issue of Homeland Security Solutions, it was reported, “The anthrax letter attacks of 2001, and subsequent deaths of five people, brought home the reality of bioterrorism to Americans and provided a wake-up call for the U.S. government about the need for a method to detect and mitigate the impact of any such future attacks. Long before the anthrax letter attacks, scientists at two of the U.S. Department of Energy's national laboratories. Lawrence Livermore National Laboratory (LLNL) and Los Alamos National Laboratory (LANL), were busy pioneering a “biodetector” akin to a smoke detector to rapidly detect the criminal use of biological agents.”

The new technology is expected to play a large role in the U.S. government's homeland security counter-terrorism initiative, Bio-Watch, which is designed to detect airborne bioterrorist attacks on major U.S. cities within hours. The system, an Autonomous Pathogen Detection System (APDS), is file-cabinet-sized machine that sucks in air, runs tests, and reports the results itself. APDS integrates a flow cytometer and real-time PCR detector with sample collection, sample preparation, and fluidics to provide a compact, autonomously operating instrument capable of simultaneously detecting multiple pathogens and/or toxin. APDS provides the ability to measure up to 100 different agents and controls in a single sample.

However, there exists a significant need for smaller devices that are portable or are amenable to point-of-care use in hospitals, emergency rooms or clinics and are capable of detecting multiple targets, e.g. pathogens rapidly and accurately.

### SUMMARY OF THE INVENTION

The invention is directed generally to a system comprising a reagent chamber, a sample chamber, a sample preparation chamber, a thermocycler comprising a conductive copper section defining a cavity for receiving a sample, wherein said thermocycler is operatively connected to said cavity, a flow cytometer, at least one channel connecting said reagent chamber to said sample preparation chamber and at least one channel connecting said sample chamber to said sample preparation chamber, at least one channel from said sample preparation chamber to said thermal cycler and at least one channel from said thermal cycler to said flow cytometer.

In addition, the system further includes at least one fluid pump and may also include a waste chamber. In a preferred embodiment, the system does not include a hybridization chamber downstream of said thermal cycler.

In addition, the invention is directed to a nucleic acid assay apparatus for analyzing a sample using a reagent. The apparatus includes a thermal cycler comprising a copper section defining a cavity for receiving sample, a sample and reagent delivery unit operatively connected to said thermal cycler for delivering the sample and the reagent to said thermal cycler, a bead trap operatively connected to said thermal cycler, and a flow cytometer operatively connected to said bead trap. In addition, the system further includes at least one fluid pump and may also include a waste chamber. In a preferred embodiment, the system does not include a hybridization chamber downstream of said thermal cycler.

Also included in the invention is a method of using the aforementioned systems and apparatus. The method includes providing the above described systems, transporting the sample and the reagent to said thermal cycler for amplification, and analyzing the sample with said flow cytometer operatively connected to said thermal cycler.

These and other advantages of the invention will become more apparent from the following detailed description when taken in conjunction with the accompanying exemplary drawings.

### BRIEF DESCRIPTION OF THE SEVERAL VIEWS OF THE DRAWINGS

FIG. 1 is a fluidic diagram that illustrates one embodiment of a nucleic acid analyzer of the present invention.

FIG. 2 shows additional details of the reagent delivery system of the hybrid nucleic acid analyzer of FIG. 1.

FIG. 3 shows additional details of the thermal cycler of the hybrid nucleic acid analyzer of FIG. 1.

FIG. 4 shows additional details of the flow cytometer of the hybrid nucleic acid analyzer of FIG. 1.

FIG. 5 shows exemplary beads used in the hybridization chamber and flow cytometer of FIG. 1.

FIG. 6 illustrates how the beads are used in the hybridization chamber and the flow cytometer described in FIG. 1.

FIG. 7 illustrates how the beads are analyzed in the flow cytometer of the system.

### DETAILED DESCRIPTION OF THE INVENTION

Given the significant need to develop smaller diagnostic devices that provide rapid, and accurate multiplex analyses of samples, the present invention provides an improved integrated multiplex analysis device. By “multiplex” is meant the ability to simultaneously or substantially simultaneously analyze a sample for multiple targets, for example target nucleic acid sequences, or to simultaneously or substantially simultaneously analyze multiple samples. Accordingly, multiplex analysis can include the simultaneous analysis of at least 10 targets or samples. More preferably it includes the simultaneous analysis of at least 100 or even at least 500 targets or samples. Even more preferably it includes the simultaneous analysis of least 1000 or even 5000 or even 10,000 targets or samples.

Accordingly, the present invention provides an apparatus for multiplex analysis of samples or targets. The apparatus of the invention includes sample and reagent reservoirs operatively connected to a thermal cycler. The thermal cycler is operatively connected to a detector that in preferred embodiments includes a flow cytometer to detect encoded microspheres. In alternative embodiments, the present apparatus includes an optional bead trap downstream of the thermal cycler and upstream of the detector. The bead trap is preferentially used for washing the beads/microspheres prior to analysis in the detector. In contrast to similar devices of the prior art, the present apparatus does not include a hybridization chamber downstream of the thermal cycler.

Previously, the hybridization chamber was used for hybridization of the amplicons produced in the thermal cycler to microspheres. By “amplicons” is meant the amplified nucleic acid product resulting from the amplification reaction. In contrast, in the present device, such hybridization occurs in the thermal cycler itself. As such, by reducing the number of discrete components as compared to previous devices, e.g. removing the hybridization chamber, the present device is notably smaller than previous devices. That is, the present device is much more easily miniaturized as compared to devices in the prior art.

A significant benefit of the present device is that a separate hybridization chamber is not included in the device. As noted above, hybridization of the amplicons to microspheres occurs in the thermal cycler itself. However, such a configuration flies in the face of conventional wisdom which would suggest that time in a thermal cycler should be minimized and certainly one should not increase flow of reagent and/or components into the thermal cycler. The skilled artisan would appreciate that any increase in time, sample and/or reagents in the thermal cycler significantly increases the likelihood of contamination of the thermal cycler.

However, according to the present invention, this type flow into the thermal cycler is what is required to add microspheres to the thermal cycler for hybridization. That is, the previous devices moved amplicons away from the thermal cycler to a separate hybridization chamber while the present invention moves microspheres into the thermal cycler. For example, when sample is moved into the thermocycler, the thermocycler is empty. A zone of fluid containing sample moves into the chamber. The zone of fluid can be flanked by air gaps. In this embodiment, the back end of the zone is bracketed by an air gap. Following amplification, a fluid zone containing beads is moved into the thermocycler. The sample fluid zone is compressed against the air zone, but the two fluid zones can mix, all within the thermocycler. That is, with a compressed air zone, and the mixture of the two fluids, all can be contained within the thermocycler. In certain embodiments, beads are only moved into the thermocycler after amplification is complete, and are heated to ensure separation of the nucleic acid strands, and facilitate maximum hybridization to the probes on the beads. In preferred embodiments the beads are heated at least once. Preferably, the beads are heated less than 10 times with less than 5 times being preferred. Even more preferred is a method wherein the beads are heated less than about 2 times or once.

An additional surprising discovery is that the improved device allows for increased sample or amplicon recovery because there are simply fewer components in contact with the sample, e.g. fewer surfaces to which the sample or amplicons may non-specifically bind. This results in increased signal-to-noise ratios, which results in increased assay sensitivity. This provides a system where detection of false positives and/or false negatives is substantially reduced.

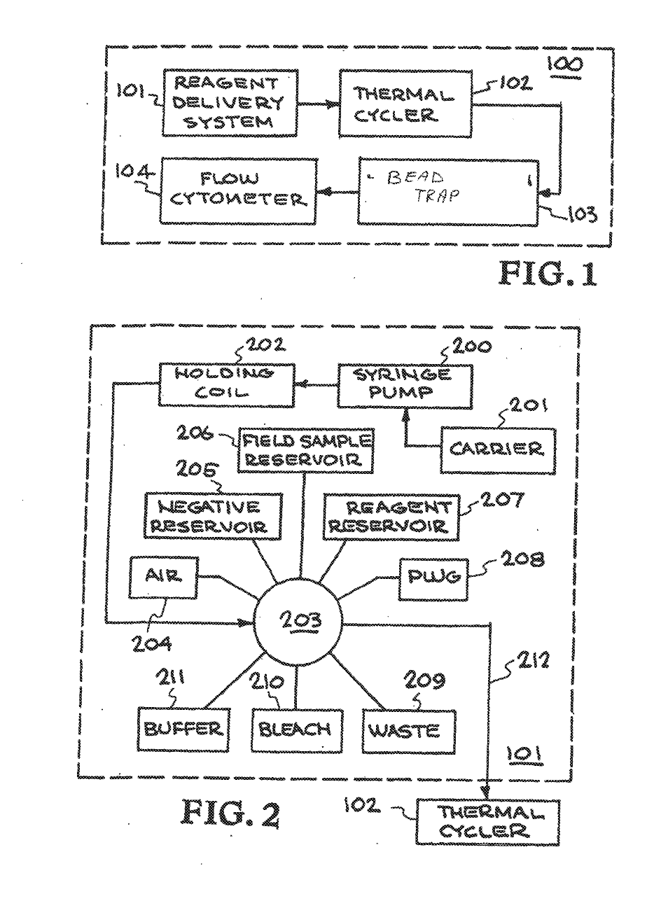

A preferred apparatus of the invention is shown in FIG. 1. FIG. 1 depicts a fluidic diagram of one embodiment of a hybrid nucleic acid analyzer. This embodiment of the hybrid nucleic acid analyzer is designated generally by the reference numeral 100. Nucleic acid and protein analyses are usually performed on separate analysis platforms.

The hybrid nucleic acid analyzer system 100 comprises a reagent delivery system 101, a thermal cycler 102, optionally a bead trap for washing 103, and a flow cytometer 104. In a particularly preferred embodiment the system does not include a discrete hybridization chamber where microspheres are hybridized with amplicons.

Although the prior art recognized the desire to include a separate hybridization chamber for hybridization of beads to the amplicons, the present invention performs this hybridization in the thermal cycler itself. That is, following amplification the microspheres are brought into the thermal cycler for hybridization with the amplicons. The advantage of doing so is that a separate component is, e.g. the hybridization chamber, is eliminated from the apparatus. This results in a less complicated system that provides for miniaturization. In addition, having fewer chambers in the system results in increased efficiency as compared to the apparatus with the hybridization chamber. That is, it has been found by the present invention that having fewer components allows for maximal sample recovery at each step because there are fewer manipulations with the sample and the sample is contacted with fewer surfaces. In addition, because of the improved recovery, the system of the invention lacking the hybridization chamber allows for increased signal-to-noise ratios. Without being bound by theory, it is thought that the improved recovery improves the overall signal strength.

In some embodiments, when desirable, an in-line bead-trap 103 follows the thermal cycler. In this embodiment the beads are moved from the thermal cycler to the bead trap where they undergo washing prior to being analyzed in the flow cytometer.

In one embodiment the device of the invention includes a sample preparation chamber. Preferably this chamber is operatively connected to the sample chamber and one or more reagent chambers. Upon initialization of a run or assay, sample and reagent are brought into this chamber where necessary sample preparation, for example, clarifying the sample or extracting nucleic acids, is carried out. Examples of sample preparation chambers or cartridges is found in U.S. Pat. No. 6,881,541, which is expressly incorporated herein by reference. In embodiments that include sample preparation chambers, the sample preparation chamber is operatively connected to the thermal cycler. After sample preparation, the sample is transported, e.g. pumped, into the thermal cycler.

The reagent delivery system 101 delivers PCR reagents to the thermal cycler 102 autonomously. On completion of cycling in the thermal cycler 102, beads or microspheres are added to the sample in the thermal cycler. The hybridized beads are then moved to the flow cytometer 104 for analyses. Alternatively, the hybridized beads are added to the bead trap 103 where they undergo washing prior to being moved to the flow cytometer.

The hybrid nucleic acid analyzer system 100 utilizes nucleic acid amplification and detection and sample preparation and analysis techniques and information described in currently co-pending U.S. patent application Ser. Nos. 10/189,319 and 10/643,797, both of which are owed by the Regents of the University of California, the assignee of this application. U.S. patent application Ser. No. 10/189,319 for an “Automated Nucleic Acid Assay System” was filed Jul. 2, 2002 by Billy W. Colston, Jr., Steve B. Brown, Shanavaz L. Nasarabadi, Phillip Belgrader, Fred Milanovich, Graham Marshall, Don Olson, and Duane Wolcott and was published as U.S. patent application No. 2003/0032172 on Feb. 13, 2003. U.S. patent application Ser. No. 10/643,797 for a “System for Autonomous Monitoring of Bioagents” was filed Aug. 19, 2003 by Richard G. Langlois, Fred Milanovich, Billy W. Colston, Jr., Steve B. Brown, Don A. Masquelier, Ray P. Mariella, and Kodomundi Venkateswaran and was published as U.S. patent application No. 2004/0038385 on Feb. 26, 2004. The disclosures of U.S. patent application Ser. Nos. 10/189,319 and 10/643,797 are incorporated herein by this reference.

A LabView interface software system controls the fluidic handling and the operation of the thermal cycler 102. The LabView interface software system software is integrated into a form compatible with the Graphical User Interface (GUI) used to control and monitor the flow cytometer 104.

The system software leverages developments made for two autonomous environmental monitoring systems developed at LLNL; RAIDDS and APDS. For example, FluIDₓ uses the same drivers, step-types (script elements), routine execution engine, and most of the support architecture used in RAIDDS and APDS, including error handling and logging. However, there are differences between the software architecture used these systems. The APDS/RAIDDS software (Ver 1.x) was designed and modified to meet the highly-specialized and specific requirements of each of these two instruments. FluIDₓ will utilize the next-generation software (Version 2.x), which has been generalized such that it can be adapted to any application. Primary differences between Ver 1.x and Ver 2.x include the removal of legacy code and the addition of customization capabilities. Version 2.x software was developed with modularity in mind, which makes the changes very transparent to the user. Many of the changes involve software flow and organization.

The system will have an “Operator” screen that is distinct from either RAIDDS or APDS. The Operator screen will show the state of the system (on, off, running, where in the process the instrument is), a list of the routines that can be run, results of the previous run and have several option buttons. There will be buttons to start a run, enter data, print a report, or login for advanced operations. Note: there will be no login required for using the “Operator” level functions. Initially, there will be a login for using the “Developer” functions which include routine editing, manual operation, configuration editing, etc.

The ISDAT software is capable of handling changes to hardware with simple changes to a configuration file. This file instructs the software what port to or line to use for individual sub-systems. This allows drivers originally written for one project to be immediately useable on another project by simply indicating which port the device is now using on the new project.

The primary operational mechanism is the routine. This is a ‘script’ for executing various commands in any order desired by the designer. The routine is built and tested by an expert. The designer is able to set the order in which individual components of the system receive commands. These routines, once tested and verified, can be used by any user by simply selecting the routine and hitting “run”. The routines that are available for “operators” are selected by the designers and appear in a list on the “operator” screen. A typical list includes: main routine, cleanup, initialization, calibration, etc. This gives the user high level options without the need to understand the effects of each command in the routine.

The software already has a capability for handling algorithms with a step-type and a configuration page. These will need to be modified to handle the new algorithm being developed. The person doing the new algorithm knows the form the algorithm needs to be in to function within the ISDAT code.

The ISDAT code is setup to communicate with outside entities. The preferred mechanism is via Ethernet. This is the most reliable and readily available communications media. This allows for wireless (WiFi, or cellular), wired (any Ethernet will do) or even within the same computer. The APDS communications system uses Virtual Private Networking (VPN) and a customized low bandwidth protocol to communicate with its central data server.

During analysis, the detector counts the instances a given bead appears in the course of that sample analysis. The detector records the fluorescent intensity of each classified and counted bead. Thus, each bead type will have a distribution of fluorescence values associated with it. To quantify the response of each bead type in an individual assay, the distribution of fluorescence is summarized by its median value. Thus, each assay results in median fluorescent intensity (MFI) value for each bead type within the assay. The results of the assay are evaluated (deemed positive or negative) based upon these MFI signals.

The interpretation of the data begins with a determination of the validity of the data by evaluating the MFI values of the internal assay controls. Each control is evaluated with respect to a MFI value which is pre-determined based on assays run during the development of the instrument. If the MFI values for the control beads fall below this predetermined value (or in the case of a negative control, rise above the pre-determined value), the results of the assay are suspect, and thus, the MFI signals cannot be evaluated with respect to determining whether a given signal is positive or negative. If all of the controls are within their acceptable ranges, a call of positive or negative can be made.

To interpret the MFI values with respect to being positive or negative, each signal from each bead type is again compared to a threshold value. These threshold values are determined by establishing a rate at which blank samples are correctly ruled as a negative. This requires measuring the MFI of each bead type for a large number (˜1000) of blank samples and generating a histogram describing the distribution of the resulting MFI values for each individual signature. From each histogram, a function can be derived describing the relationship between a threshold value and the rate at which false positives occur. After an acceptable rate of false positives is selected, the MFI threshold for each bead type can then be determined from this function. When during the course of an assay, a bead reports an MFI signal above its associated threshold value, that assay would be ruled positive, otherwise the assay is ruled negative.

Referring now to FIG. 2, additional details of the reagent delivery system 101 of the hybrid nucleic acid analyzer system 100 are shown. The reagent delivery system 101 includes a syringe pump 200 that delivers a carrier 201 to a holding coil 202. The carrier is available to a zone fluidics system. The zone fluidics system provides sequential injection analysis (SIA).

Zone fluidics defines a general-purpose fluidics tool, allowing the precise manipulation of gases, liquids and solids to accomplish very complex analytical manipulations with relatively simple hardware. Zone fluidics is the precisely controlled physical, chemical, and fluid-dynamic manipulation of zones of miscible and immiscible fluids in narrow bore conduits to accomplish sample conditioning and chemical analysis. A zone is a volume region within a flow conduit containing at least one unique characteristic.

A unit operation in zone fluidics comprises of a set of fluid handling steps intended to contribute to the transformation of the sample into a detectable species or prepare it for manipulation in subsequent unit operations. Examples of unit operations include sample filtering, dilution, enrichment, medium exchange, headspace sampling, solvent extraction, matrix elimination, de-bubbling, amplifying, hybridizing, and reacting. In current analytical practice many of these steps are handled manually or in isolated pieces of equipment. Integration is scant at best, and there is a high degree of analyst involvement. In zone fluidics, sample and reagent zones are subjected to these unit operations in a sequential manner being transported from one unit operation to the next under fluidic control.

Zone fluidics provides an alternative approach whereby unit operations are performed in narrow bore conduits and the transportation medium, instead of being mechanical as in robotics, is fluidic. At the heart of a zone fluidics manifold is a multi-position selection valve. Fluids are propelled and manipulated in the manifold by means of a bi-directional flow pump. A holding coil between the pump and valve is used to stack zones and mix adjacent zones through dispersion and diffusion as is practiced in sequential injection analysis (SIA).

The ports of the multi-position valve are coupled to various reservoirs, reactors, manifold devices, and detectors as indicated. Narrow bore conduits comprise the flow channels and provide fluid contact between manifold devices and components. The term fluid refers to liquids, gases, aerosols, and suspensions. Samples in zone fluidics are not limited to liquids. Rather, gases, and suspensions containing solids or cells are also included. Where solid samples are used, particles are limited to a size that ensures no blockages.

In most cases, reagents are prepared and then coupled to the zone fluidics manifold. The metering capability of the pump and mixing unit operations allow for reagents and standards to be prepared in situ. Reagents can therefore be presented to the zone fluidics manifold in an appropriately designed cartridge as ready-made, reagent concentrates, lyophilized, or crystalline form. Standards can be plumbed to the multi-position valve as discrete reservoirs providing the required range of concentrations. As for reagents though, standards can also be prepared in situ or diluted to cover a larger dynamic range.

In the reagent delivery system 101, a syringe pump 200 delivers a carrier 201 to a holding coil 202. The carrier is available to a zone fluidics system. The zone fluidics system provides in sequential injection analysis (SIA). The ports of a multi-position valve 203 of the zone fluidics sequential injection analysis system are coupled to air reservoir 204, negative reservoir 205, field sample reservoir 206, reagent reservoir 207, plug 208, waste 209, bleach reservoir 210, and bleach reservoir 211 as indicated. The zone fluidics sequential injection analysis system has an outlet 212 that delivers PCR reagents to the thermal cycler 102.

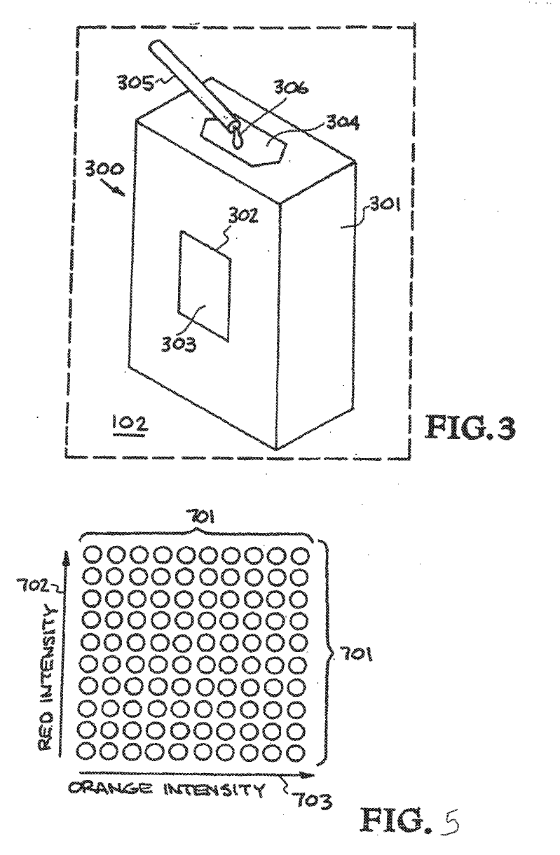

Referring now to FIG. 3, details of the thermal cycler 102 of the hybrid nucleic acid analyzer system 100 of the present invention are shown. Currently available Polymerase Chain Reaction (PCR) thermal cycling units are large cumbersome and non-portable. Some examples of commercially available semi-portable instruments include the iCycler manufactured by Bio-Rad, the Light cycler from Idaho Technologies and the Smart Cycler from Cepheid Inc. In addition to amplification reactions described above, real-time PCR works by including in a reaction mix sequence specific oligonucleotides (primer) that can be extended at its 3′ end and a third non-extendable oligonucleotide (probe) that has two fluorescence molecules attached to its 5′ and 3′ end respectively. Thus the probe is quenched due to the Fluorescence Resonance Energy Transfer (FRET) between the two fluorescent molecules. FRET is dependent on the sixth power of the intermolecular separation of the two fluorophores. In the absence of primer extension, there is no fluorescence signal detected by the fluorimeter. The enzyme DNA polymerase has 5′-3′ exo-nuclease activity as well as 5′-3′ polymerase activity. During primer extension, the fluorophore is cleaved from the 5′ end of the probe and since the fluorophore is no longer quenched, a signal is detected by the fluorimeter. These instruments are designed for measuring the fluorescence released from sequence specific probes in case of a positive identification. At present the multiplexing of nucleic acid signatures is limited by the number of fluorophores that can be used in the commercial instruments, due to spectral overlap of most of these fluorophores.

The thermal cycler 102 of the present invention can be a unit such as that described in U.S. Pat. No. 5,589,136 issued Dec. 31, 1996 to M. Allen Northrup, Raymond P. Mariella, Jr., Anthony V. Carrano, and Joseph W. Balch and assigned to the Regents of the University of California or in U.S. Pat. No. 6,586,233 issued Jul. 1, 2003 to William J. Benett. James B. Richards, and Fred P. Milanovich and assigned to the Regents of the University of California. The disclosures of U.S. Pat. No. 5,589,136 issued Dec. 31, 1996 and U.S. Pat. No. 6,586,233 issued Jul. 1, 2003 are incorporated herein by this reference. In addition, the thermal cycler as set forth in U.S. application Ser. No. 10/272,178 (US PGPUB 20040072334), to Benett et al., which is expressly incorporated herein by reference, finds use in this invention. This thermal cycler is particularly useful as the chamber units are preferably made from copper. Copper provides good thermal conductivity. This is described in more detail in US PGPUB 20040072334).

The thermal-cycler allows the controlled heating and cooling of the sample to perform the PCR amplification. The thermal-cycler is a flow-through chamber made from two photo-lithographically patterned and etched copper plates. The etched channel on the inside of the chamber allows the sample tubing to be clamped between the two chamber halves, insuring good thermal contact. The tubing is connected directly to the fluidics system allowing the sample to be moved into and out of the thermal-cycler. The etched features on the outside of the chamber create increased surface area to enhance forced convective cooling.

Heating is accomplished by clamping the chamber between two circuit board assemblies. Standard surface mount resistors soldered to the circuit boards act as heaters. A surface mount linear thermistor provides temperature sensing for control of the thermal-cycling. During the cooling cycle, air is forced through slots in the circuit board assembly, onto the chamber.

As show in FIG. 3, a chamber unit 300 is fabricated of circuit board material. The system can be constructed of materials such as circuit board fiberglass, silicon, ceramics, metal, or glass. Advantages of using circuit board fiberglass is the fact that it is not as thermally conductive as the other materials and the heating is more efficiently applied to the sample rather than being conducted to surrounding materials. Circuit board material is readily available and the technology of producing and working with circuit board material is highly developed. Circuit board material provides lower cost techniques for fabrication. Printed circuit board technology incorporates photolithography, metal etching, numerically controlled machining, and layering technologies to produce the desired device.

As shown in FIG. 3, the thermal cycler 102 is generally indicated at 300. The thermal cycler 102 includes a silicon-based sleeve as a chemical reaction chamber, generally indicated at 301, constructed of two bonded silicon parts, and which utilizes doped polysilicon for heating and bulk silicon for convective cooling, as described in greater detail hereinafter. The sleeve 301 includes a slot or opening 304 into which reaction fluid, indicated at 306, from a conduit 305 is inserted into the reaction chamber. The conduit 305 is constructed of plastic, for example, or other material which is inert with respect to the reaction mixture, thereby alleviating any potential material incompatibility issues. The sleeve is also provided with an opening 302 in which is located an optical window 303, made, for example, of silicon nitride, silicon dioxide, or polymers. The silicon sleeve reaction chamber 301 includes doped polysilicon for heating and bulk silicon for convective cooling, and combines a critical ratio of silicon and silicon nitride to the volume of material to be heated (e.g., liquid) in order to provide uniform heating, yet low power requirements.

The thermal cycler 102 can be used to rapidly and repetitively provide controlled thermal cycles to the reaction mixture. The thermal conductivity properties of the silicon or similar semiconducting substrate, help speed up the thermal rise and fall times, and allow low power operation. While silicon is unique in its thermal properties, i.e., high thermal conductivity, a combination of silicon, silicon nitride, silicon dioxide, polymers and other materials would provide a combination of thermal conductivity and insulation that would allow thermal uniformity and low power operation.

The Sample and the nucleic acid reaction mix are introduced into the thermal cycler 102 by the Sequential Injection Analysis fluid handling system illustrated in FIG. 2. As the sample is continuously driven by convection through the channels it passes through sections of channel that are temperature controlled to be at the upper and lower temperatures required for the PCR reaction. This continuous flow through the PCR temperature zones effectively thermally cycles the sample.

Referring now to FIG. 4, additional details of the flow cytometer 104 of the hybrid nucleic acid analyzer system 100 are shown. The flow cytometer 104 comprises a Luminex® LX100 Flow Cytometer instrument 600 with a sheath source 601 and a waste reservoir 602. The hybridized bead array from the beat trap 103 is introduced into the Luminex® Flow Cytometer instrument 600 where the beads are interrogated by two lasers, a red laser for the internal discriminator and a green laser for the external discriminator dyes respectively. Additional details of the flow cytometer 600 and its operation are show in the figures.

In order to multiplex more than four signatures, Applicants have designed a Luminex® Bead based Array analyzer. With this liquid arrays it is possible to multiplex at least 100 different organisms or targets. The discrimination of the polystyrene Luminex® bead array is dependent on the precise ratio of two internal discriminator dyes, a red and an infrared dye. The signal intensity on the surface of the bead is dependent on the concentration of the analyte in solution, in our case the amplified DNA of a suspect agent or an antigen or a toxin, whichever the case may be.

However, there are other encoding technologies that find use in this invention. Thus, the number different organisms or targets can exceed 100 depending on the encoding technology. That is, the number of multiplexed assays depends on the number of distinguishable bead populations. While the Luminex® system above can discriminate at least 100 bead or microsphere populations, when other bead encoding technologies are included this number increases.

There is no upward limit to the number of populations of microspheres or capture probes when populations are analyzed individually. When multiple sets of microspheres are mixed and analyzed the number of sets is limited only by the number of encoding moieties applied to the microspheres. That is, microspheres are encoded so that the identity of each set of microspheres can be determined.

Encoding moieties can be any distinguishable characteristic, e.g. size, shape, texture etc., of the microsphere. In preferred embodiments, encoding moieties are attributes that are not inherent in the bead or microsphere itself. Rather, the encoding moiety is a feature that is added to a bead. Preferred encoding moieties include, but are not limited to nucleic acids, proteins, and detectable labels or fluors. In addition, materials such as nanocrystals can be used as encoding moieties.

Also, in some embodiments, a plurality of different types of encoding moieties can be used to develop numerous different codes. Thus, the number of codes, e.g. the number of distinguishable bead populations is at least 100 with more than 500 being particularly preferred. In an even more preferred embodiment the number of distinguishable microsphere populations is at least 100, with more than 5000 or even more than 10,000 being particularly preferred.

In a preferred embodiment, the beads and encoding system are those used in the Luminex® flow cytometer, described above. This system is also described in more detail in U.S. Pat. No. 5,981,180, which is expressly incorporated herein by reference.

Referring now to FIG. 5, the beads of the invention are illustrated. A 100-plex Luminex® liquid array 700 is generated by intercalating varying ratios of red and orange infrared dyes into polystyrene latex microspheres or beads 701. The process of producing varying ratios of red and orange infrared dyes in the beads 701 is accomplished by increasing the amount of red dye as illustrated by the arrow 702 and increasing the amount of orange dye as illustrated by the arrow 703. This gives each optically encoded bead 700 a unique spectral address.

Referring now to FIG. 6, additional information is provided illustrating how the beads are used in the flow cytometer 104. The beads designated by the reference numeral 800 are coated with capture antibodies specific for target antigens or capture probes complementary to adapter sequences as described herein. Each bead has an attachment site specific for a bioagent. For example, the upper bead has an attachment site 801 for influenza nucleic acids or a capture probe capable of hybridizing with an adapter attached to a primer specific for the influenza nucleic acids. The next bead has an attachment site 802 for an adenovirus nucleic acid or a capture probe capable of hybridizing with an adapter attached to a primer specific for the adenovirus nucleic acid, and the like.

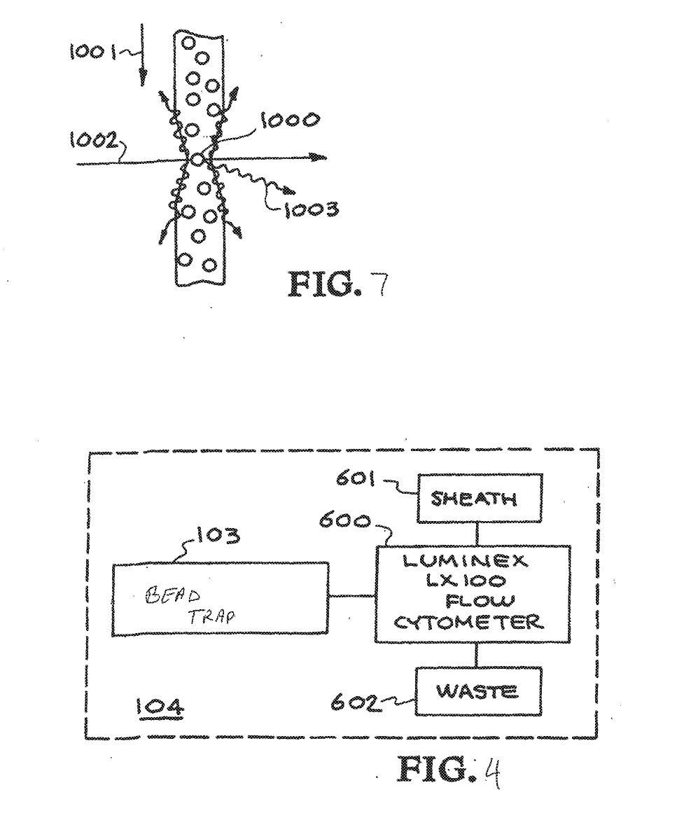

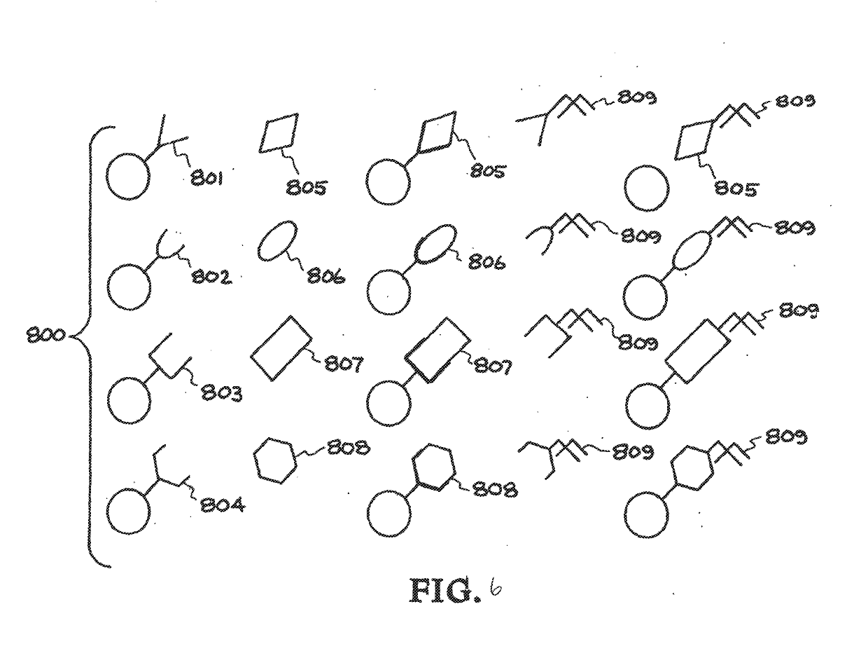

Referring now to FIG. 7, an illustration shows how the beads are analyzed in the flow cytometer. The beads are designated by the reference numeral 1000. The direction of flow is shown by the arrow 1001. The beads 1000 are interrogated one at a time. As illustrated, one bead 1000 is shown being interrogated. A red laser classifies the bead 1000, identifying the bead type. Subsequently a green laser 1002 quantifies the assay on the bead surface—only those beads with a complete sandwich will produce a fluoresce 1003 in the green, and the signal is a function of label concentration, which is indicative of the amount of target.

The hybrid nucleic acid analyzer provides an integrated nucleic acid and protein/toxin detection system capable of in-line analysis of a complex sample within an hour or less. The hybrid nucleic acid analyzer has the capability of performing continuous nucleic acid and immunoassays in a multiplex format. The hybrid nucleic acid analyzer is a field deployable instrument for detection of pathogens and toxins in environmental or clinical samples. The hybrid nucleic acid analyzer takes advantage of the multiplexing capability of the Luminex® Bead arrays complexed with multiplexed nucleic acid and protein capability developed at the Lawrence Livermore National Laboratory.

The hybrid nucleic acid analyzer has an integrated PCR chamber 102, and Luminex® LX100 flow cytometer 104 and optionally a microsphere wash chamber 103 controlled by a LabView interface software for the fluidic handling and the operation of the PCR chamber. The software is integrated into a form compatible with the Graphical User Interface (GUI) used to control and monitor the Luminex® LX100 flow cytometer. Control and data analysis software routines have been written for controlling the Luminex® LX100 flow cytometry. Provisions have been made for the addition of a sample preparation and concentration unit as well as a bead sequestering unit in order to facilitate deep multiplexing of the agents. A sample preparation and concentration strategy involves the use of Silicon pillar chips capable of handling volumes of up to 100 ml or more of the sample, releasing the DNA from the cells through lysis and concentrating it in a small volume for analysis, thus increasing the detection limit many fold.

The fluidics in the instrument is self-contained in order to minimize contamination of the surroundings and the operator. This minimizes contamination of reagents and samples, a feature not available in commercial units. The sample and the nucleic acid reaction mix are introduced into the thermal cycler 102 by Sequential Injection Analysis fluid handling system 101.

Once the sample is introduced into the instrument 100, the detection is autonomously done following the sequence of events input by the researcher. Decontamination of the fluidics system is carried out autonomously after each amplification step. The system including the PCR chamber 102, the tubing carrying the sample to the PCR chamber and all the tubing and fitting downstream from there on are rinsed with 5% household bleach which we have found sufficient to effectively remove all traces of nucleic acids or PCR product from the housing. After every PCR run, a negative control for the agent/agents is amplified in order to determine the efficacy of the decontamination process.

Manual labor is the major factor for the high cost of sample testing. The software has the capability of stacking a series of fluidic protocols for autonomous analysis. Thus the instrument 100 can be loaded with the reagents and the samples at the beginning of the day and the results can be accessed from a remote location. This cuts the cost of labor as compared to the conventional way of doing analysis. Thus with this instrument it is possible to perform continuous analysis of samples from a known set of reagents with minimal intervention in effect significantly reducing the cost of the assay.

The hybrid nucleic acid analyzer 100 provides autonomous use of both the thermal cycler 102 and the flow cytometer 104 such that protein analysis can be performed independent of the nucleic acid detection. For detection of antigens or toxins, the sample is introduced directly to appropriately labeled beads followed by hybridization to the secondary antibody and analysis of the assay in the flow cytometer 104. The hybrid nucleic acid analyzer 100 can be repeatedly decontaminated in between runs with a solution of 5% household bleach.

The nucleic acid detection is done by hybridization of the amplified PCR product with the probes attached to the surface of the bead sets, e.g. via NHS ester linkage chemistry. The PCR product is labeled with Biotin molecules and the hybridization of the product to the beads is followed by streptavidin phycoerythrin addition to the hybridized reaction mix.

The hybrid nucleic acid analyzer system 100 provides a closed integrated rapid Real-time PCR and multiplex flow analysis instrument for identification of multiplex pathogen and toxin within an hour with minimal exposure to the technician. The hybrid nucleic acid analyzer system 100 combines Real-time flow through PCR with an inline flow cytometer to detect both nucleic acids as well as proteins. Sequential injection analysis (SIA) fluidic system is used to deliver the sample and reagent for in-line mixing, analysis and archiving of samples.

The unused PCR reaction mix is moved to the waste stream. The hybrid system is decontaminated and made ready for another round of amplification by rinsing with a 5% solution of Household Bleach followed by water rinse. A negative reaction with water substituted for sample is run between sample amplifications to ensure that the system is free of carry over PCR product.

The hybrid nucleic acid analyzer 100 has many uses. For example, the system 100 has use for clinical analysis of blood bank samples in a continuous 24/7 analysis of pathogens. The system 100 has use in diagnostic labs. The system 100 has use as a fly away lab or integrated into continuous monitoring of environmental samples for detection of Biothreat agents. The system 100 also has use in automated processing, amplification and detection of biological molecules in forensic samples. The system 100 can also be used for automated clinical testing, analysis and archiving in event of an outbreak. The system 100 can also be used to detect proteins and toxins both in the clinic as well as from the environment.

All references cited herein are incorporated by reference in their entirety.

While the present invention has been described with reference to what are considered to be the specific embodiments, it is to be understood that the invention is not limited to such embodiments. To the contrary, the invention is intended to cover various modifications and equivalents included within the spirit and scope of the appended claims.

1. A nucleic acid assay apparatus for analyzing a sample using a reagent, comprising:
a thermal cycler comprising a copper section defining a cavity for receiving the sample;
a sample and reagent delivery unit operatively coupled to said thermal cycler for delivering the sample and the reagent to said thermal cycler;
a flow cytometer operatively coupled to said thermal cycler, wherein said thermal cycler is coupled between said sample and reagent delivery unit and said flow cytometer; and
a plurality of beads located upstream of the thermal cycler, wherein each bead comprises a unique attachment site specific for a bioagent nucleic acid or specific for an adaptor attached to a primer specific for the bioagent nucleic acid,
wherein said apparatus does not include a hybridization chamber downstream of the thermal cycler.

2. The apparatus of claim 1, wherein said beads comprise polystyrene latex microspheres beads with each bead having a unique spectral address.

3. The apparatus of claim 1, wherein said beads comprise beads with varying ratios of red and orange dyes giving each bead a unique spectral address.

4. The apparatus of claim 1, wherein said beads comprise a 100-plex array of beads with varying ratios of red and orange dyes giving each bead a unique spectral address.

5. The apparatus of claim 1, further comprising a bead trap, wherein said bead trap is operatively coupled to said thermal cycler and said flow cytometer.

6. The apparatus of claim 1, further comprising a sample preparation chamber operatively coupled to said sample and reagent delivery unit.

7. The apparatus of claim 1, wherein said sample and reagent delivery unit further comprises a sample reservoir operatively coupled to said sample and reagent delivery unit, a reagent reservoir operatively coupled to said sample and reagent delivery unit, and a bead reservoir operatively coupled to said sample and reagent delivery unit.

8. The apparatus of claim 1, wherein said sample and reagent delivery unit comprises a zone fluidics manifold.

9. The apparatus of claim 1, wherein said sample and reagent delivery unit comprises a sequential injection analysis system.

10. The apparatus of claim 1, further comprising at least one waste chamber operatively coupled to said sample and reagent delivery unit.

11. The apparatus of claim 1, further comprising at least one fluid pump operatively coupled to said sample and reagent delivery unit.

12. The apparatus of claim 1, further comprising a software system for controlling the sample and reagent delivery unit and the thermal cycler.

13. The apparatus of claim 1, further comprising a graphical user interface configured to control the flow cytometer.

14. The apparatus of claim 1, further comprising an Operator screen.

15. The apparatus of claim 1, further comprising an Ethernet connection.

16. The apparatus of claim 1:
said sample and reagent delivery unit comprising a zone fluidics manifold comprising a sequential injection analysis system;
further comprising a sample reservoir operatively coupled to said sample and reagent delivery unit, a reagent reservoir operatively coupled to said sample and reagent delivery unit, and a bead reservoir operatively coupled to said sample and reagent delivery unit;
said plurality of beads comprising polystyrene latex microsphere beads in the bead reservoir with each bead having a unique spectral address comprising varying ratios of red and orange dyes and a unique attachment site specific for a bioagent nucleic acid or specific for an adaptor attached to a primer specific for the bioagent nucleic acid;
further comprising a bead trap for washing beads operatively coupled to said thermal cycler and said flow cytometer;
further comprising at least one waste chamber operatively coupled to said sample and reagent delivery unit;
further comprising at least one fluid pump operatively coupled to said sample and reagent delivery unit; and
further comprising a software system for controlling the delivery unit and the thermal cycler, a graphical user interface configured to control the flow cytometer, an Operator screen; and an Ethernet connection.

17. A nucleic acid assay method for analyzing a sample using a reagent, comprising the steps of: providing the apparatus according to claim 1, transporting the sample and the reagent to said thermal cycler for amplification, and analyzing the sample with said flow cytometer.

18. A nucleic acid assay method for analyzing a sample, comprising the steps of:
providing the apparatus of claim 1,
providing at least one sample in the sample reservoir;
providing PCR reagents in the reagent reservoir;
delivering the sample and PCR reagents to the thermal cycler and performing nucleic acid amplification;
delivering the beads to the thermal cycler and performing nucleic acid hybridization;
delivering the beads to the flow cytometer and detecting the presence of amplified bioagent nucleic acid bound to the beads.

19. A nucleic acid assay method for analyzing a sample, comprising the steps of:
providing the apparatus of claim 16,
providing at least one sample in the sample reservoir;
providing PCR reagents in the reagent reservoir;
delivering the sample and PCR reagents to the thermal cycler and performing nucleic acid amplification;
delivering the beads to the thermal cycler and performing nucleic acid hybridization;
delivering the beads to the flow cytometer and detecting the presence of amplified bioagent nucleic acid bound to the beads.
